(19) United States
(12) Patent Application Publication
(10) Pub. No.: US 2024/0374213 A1
(43) Pub. Date: Nov. 14, 2024

(54) PROVISION SYSTEM, PROVISION METHOD, AND COMPUTER PROGRAM

(71) Applicant: Sumitomo Electric Industries, Ltd., Osaka-shi, Osaka (JP)

(72) Inventors: Tomoya MAEKAWA, Osaka-shi, Osaka (JP); Shingo HIBINO, Komaki-shi, Aichi (JP); Naoki OYAIZU, Komaki-shi, Aichi (JP); Yuka BEPPU, Komaki-shi, Aichi (JP); Atsushi UEDA, Komaki-shi, Aichi (JP)

(73) Assignee: Sumitomo Electric Industries, Ltd., Osaka-shi, Osaka (JP)

(21) Appl. No.: 18/695,842

(22) PCT Filed: Sep. 29, 2021
(86) PCT No.: PCT/JP2021/035898

§ 371 Date: Mar. 27, 2024

(51) Int. Cl. A61B 5/00; A61B 5/16

(52) U.S. Cl. CPC A61B 5/6843; A61B 5/165

(57) ABSTRACT

This provision system includes: a presentation unit configured to present, to a user, a fact that contacting of a sensor with a body surface of the user serves as a condition for permission to provide a physical quantity to another entity other than the user, the sensor having a contact surface and being configured to measure the physical quantity, the physical quantity indicating a degree of change in a surface with which the contact surface contacts; an estimation unit configured to estimate a contact state of the sensor with the body surface on the basis of the physical quantity; and a provision unit configured to start providing the physical quantity to the other entity, on the basis of an estimation result for the contact state by the estimation unit.

### TECHNICAL FIELD

The present invention relates to a provision system, a provision method, and a computer program.

### BACKGROUND ART

Conventionally, a system for analyzing transition of the emotion of an event participant from a video obtained by a camera capturing the expression of the event participant at a live venue for a concert or the like, has been developed (see, for example, NON PATENT LITERATURE 1).

In addition, an information processing system for analyzing the emotion of a user by using means such as a wearable terminal worn by the user has been proposed (see, for example, PATENT LITERATURE 1).

### CITATION LIST

### Patent Literature

PATENT LITERATURE 1: WO2017/064891

### Non Patent Literature

NON PATENT LITERATURE 1: “Avex×MS ga kankyaku no hyojo wo toraeru—entame gyokai de AI wo katuyousuru toiu chosen (Avex×MS grasps the emotions of audience—challenge of utilizing AI in entertainment industry)” [online], cnet Japan (ASAHI INTERACTIVE, Inc.), [searched on Aug. 30, 2021], the Internet <URL: https://japan.cnet.com/article/35116085/>
NON PATENT LITERATURE 2: “Thin Flexible Battery (Air Patch Battery) (Under Development)”, [online], Maxell Holdings, Ltd., [searched on Aug. 30, 2021], the Internet <URL: https://biz.maxell.com/ja/primary_batteries/air_patch_battery.html>
NON PATENT LITERATURE 3: “next-generation RFID battery-free Bluetooth (registered trademark) tag wiliot”, [online], SATO HOLDINGS CORPORATION, [searched on Sep. 22, 2021], the Internet <URL: https://www.sato.co.jp/taggingtown/wiliot.html>

### SUMMARY OF THE INVENTION

A provision system according to an aspect of the present disclosure includes: a presentation unit configured to present, to a user, a fact that contacting of a sensor with a body surface of the user serves as a condition for permission to provide a physical quantity to another entity other than the user, the sensor having a contact surface and being configured to measure the physical quantity, the physical quantity indicating a degree of change in a surface with which the contact surface contacts; an estimation unit configured to estimate a contact state of the sensor with the body surface on the basis of the physical quantity; and a provision unit configured to start providing the physical quantity to the other entity, on the basis of an estimation result for the contact state by the estimation unit.

A provision method according to another aspect of the present disclosure includes: a presentation step of presenting, to a user, a fact that contacting of a sensor with a body surface of the user serves as a condition for permission to provide a physical quantity to another entity other than the user, the sensor having a contact surface and being configured to measure the physical quantity, the physical quantity indicating a degree of change in a surface with which the contact surface contacts; an estimation step of estimating a contact state of the sensor with the body surface on the basis of the physical quantity; and a provision step of starting providing the physical quantity to the other entity, on the basis of an estimation result for the contact state.

A computer program according to another aspect of the present disclosure causes a computer to function as: a presentation unit configured to present, to a user, a fact that contacting of a sensor with a body surface of the user serves as a condition for permission to provide a physical quantity to another entity other than the user, the sensor having a contact surface and being configured to measure the physical quantity, the physical quantity indicating a degree of change in a surface with which the contact surface contacts; an estimation unit configured to estimate a contact state of the sensor with the body surface on the basis of the physical quantity; and a provision unit configured to start providing the physical quantity to the other entity, on the basis of an estimation result for the contact state by the estimation unit.

### BRIEF DESCRIPTION OF DRAWINGS

FIG. 1 shows an example of an entire configuration of an emotion analysis system according to an embodiment.

FIG. 2 is a block diagram showing an example of a configuration of each of a sensor and a server constituting the emotion analysis system according to the embodiment.

FIG. 3 shows a structure of the sensor.

FIG. 4 shows an example of arrangement positions of strain gauges at a circuit layer.

FIG. 5 shows an example of a peel-off sheet composing the sensor.

FIG. 6 is a block diagram showing an example of a configuration of a smartphone according to the embodiment.

FIG. 7 is a sequence diagram showing an example of user registration processing for registering user information on a user who uses a sensor in the server.

FIG. 8 shows an example of a user information registration screen.

FIG. 9 shows an example of user information registered in the server.

FIG. 10 is a sequence diagram showing an example of event registration processing.

FIG. 11 shows an example of an event information screen.

FIG. 12 shows an example of a sticker information screen.

FIG. 13 shows an example of a terms-of-use information screen.

FIG. 14 is a sequence diagram showing an example of linkage processing.

FIG. 15 shows an example of a QR code (registered trademark) reading screen.

FIG. 16 shows an example of a user ID input screen.

FIG. 17 shows an example of user information with sensor IDs additionally registered.

FIG. 18 is a sequence diagram showing an example of strain amount measurement processing.

FIG. 19 shows an example of temporal change in a strain amount.

FIG. 20 shows an example of temporal change in a strain amount.

FIG. 21 is a block diagram showing an example of a configuration of a smartphone according to a modification.

### DETAILED DESCRIPTION

### Problems to be Solved by the Present Disclosure

In the system described in NON PATENT LITERATURE 1, acquisition and analysis of data such as a video related to the emotion and the behavior of a user might be performed without user's permission. Therefore, some users feel unpleasant. Thus, there is a possibility that some users have a feeling of resistance such as a feeling of rejection or a feeling of fear against reading of their emotions.

In the system described in PATENT LITERATURE 1, a user needs to wear a wearable terminal such as a smartwatch, a smart band, or smart glasses. Wearing such a wearable terminal gives a feeling of strangeness to the user, and there is a possibility that the user has a feeling of resistance such as a feeling of rejection or a feeling of fear against reading of their emotions.

The present disclosure has been made in view of the above circumstances, and an object of the present disclosure is to provide a provision system, a provision method, and a computer program for analyzing an emotion without giving a feeling of strangeness and a feeling of resistance to a user.

### Effects of the Present Disclosure

According to the present disclosure, it is possible to analyze an emotion without giving a feeling of strangeness and a feeling of resistance to a user.

### Description of Embodiment of the Present Disclosure

Hereinafter, the outline of an embodiment of the present disclosure is listed and described.

(1) A provision system according to an embodiment of the present disclosure includes: a presentation unit configured to present, to a user, a fact that contacting of a sensor with a body surface of the user serves as a condition for permission to provide a physical quantity to another entity other than the user, the sensor having a contact surface and being configured to measure the physical quantity, the physical quantity indicating a degree of change in a surface with which the contact surface contacts; an estimation unit configured to estimate a contact state of the sensor with the body surface on the basis of the physical quantity; and a provision unit configured to start providing the physical quantity to the other entity, on the basis of an estimation result for the contact state by the estimation unit.

With this configuration, for example, the user can bring the sensor into contact with a body surface such as the face after approving the condition that the physical quantity obtained from the user as a measurement target is provided to another entity when the sensor is brought into contact with the body surface of the user. Thus, it is possible to prevent the physical quantity of the user from being provided to the other entity while the user does not known that fact. In addition, the user can bring the sensor into contact with the body surface with a feeling as if pasting a sticker on the face. In particular, at a sports watching venue, an attraction venue, or the like, the users have a less feeling of resistance against pasting a sticker on the face. Therefore, the other entity can collect physical quantities without giving a feeling of strangeness and a feeling of resistance to the users and thus can analyze the emotions of the users.

(2) The estimation unit may estimate that the sensor is in contact with the body surface, on the basis of a comparison result between the physical quantity and a first threshold, and when the estimation unit has estimated that the sensor is in contact with the body surface, the provision unit may start providing the physical quantity to the other entity.

When the sensor is not in contact with the body surface of the user, change in the contact surface is small and the physical quantity is also small, but for example, when the sensor is pasted on the face surface of the user, the physical quantity increases with movement of the mimic muscle or the like. Accordingly, for example, when the physical quantity is the first threshold or greater, it can be estimated that the sensor is in contact with the body surface, and provision of the physical quantity to the other entity can be started. Thus, it can be estimated that the sensor is in contact with the body surface and it is possible to prevent the physical quantity from being provided to the other entity before the sensor contacts with the body surface. Therefore, the other entity can analyze the emotion accurately.

(3) The estimation unit may estimate that the sensor is in contact with the body surface, on the basis of a period in which the comparison result between the physical quantity and the first threshold continues.

In a case where the sticker is temporarily deformed due to external pressure or the like before contacting with the body surface, the physical quantity temporarily becomes great, but the state in which the physical quantity is great does not continue. Therefore, for example, when the state in which the physical quantity is the first threshold or greater has continued during the predetermined period, it can be estimated that the sensor is in contact with the body surface.

(4) The estimation unit may estimate that the sensor is not in contact with the body surface, on the basis of a comparison result between the physical quantity and a second threshold, and when the estimation unit has estimated that the sensor is not in contact with the body surface, the provision unit may finish providing the physical quantity to the other entity.

When the sensor is not in contact with the body surface, change in the contact surface is small and the physical quantity is also small. Accordingly, for example, when the physical quantity has become smaller than the second threshold, it can be estimated that the sensor is not in contact with the body surface, and provision of the physical quantity to the other entity can be finished. Thus, it can be estimated that the sensor is not in contact with the body surface and it is possible to prevent the physical quantity from being provided to the other entity while the sensor is not in contact with the body surface. Therefore, the other entity can analyze the emotion accurately.

(5) The estimation unit may estimate that the sensor is not in contact with the body surface, on the basis of a period in which the comparison result between the physical quantity and the second threshold continues.

Even in a state in which the sensor is in contact with the body surface, in such a case where, for example, the user's face temporarily becomes expressionless, the physical quantity temporarily becomes small, but the body surface slightly vibrates. Thus, even under the expressionless face, certain change arises and the state in which the physical quantity is small does not continue. Therefore, for example, when the state in which the physical quantity is smaller than the second threshold has continued during the predetermined period, it can be estimated that the sensor is not in contact with the body surface.

(6) The provision system may further include: a registration unit configured to register a sensor identifier for identifying the sensor; and a physical quantity reception unit configured to receive, from the sensor, a set of the physical quantity and the sensor identifier of the sensor, and the provision unit may provide, to the other entity, the set including the sensor identifier registered by the registration unit among the sets received by the physical quantity reception unit.

With this configuration, it is possible to provide, to the other entity, the set of the sensor identifier registered in the provision system and the physical quantity measured by the sensor corresponding to the sensor identifier. Thus, the provision system can select the set including the registered sensor identifier from the sets of the physical quantities and the sensor identifiers received from a plurality of sensors, and provide the selected set to the other entity. For example, by registering in advance the sensor identifier of the sensor to be used by the user, it is possible to efficiently provide the physical quantity of the user to the other entity.

(7) The provision unit, further, may not transmit, to a device used by the other entity, the set not including the sensor identifier registered by the registration unit among the sets received by the physical quantity reception unit.

With this configuration, the set including the sensor identifier not registered in the provision system among the sets received by the physical quantity reception unit is not provided to the other entity. For example, if the provision system in which the sensor identifier is registered is uniquely set, the set of this sensor identifier and the corresponding physical quantity is provided from only one provision system. Thus, it is possible to prevent duplicate physical quantities from being provided to the other entity.

(8) The registration unit, further, may provide a user identifier for identifying the user and the sensor identifier, to a device used by the other entity.

With this configuration, the device used by the other entity can specify the user from the set of the physical quantity and the sensor identifier provided from the provision system, and can analyze the emotion for each user. It is noted that the user identifier is not included in the set provided from the provision system to the other entity. Therefore, even if a third party intercepts the set, the third party cannot specify the user for which the physical quantity has been measured. Thus, the privacy of the user can be protected.

(9) The provision unit, further, may provide measurement position information for the physical quantity, to a device used by the other entity.

With this configuration, the other entity provided with the measurement position information for the physical quantity can analyze the emotion for each measurement position.

(10) The provision system may further include a setting unit configured to make a setting for whether or not the user permits providing the physical quantity, and when the user does not permit providing the physical quantity, the provision unit may stop providing the physical quantity to the other entity.

With this configuration, provision of the physical quantity can be stopped by setting, even in a state in which the user has the sensor in contact with the body surface. Thus, it is possible to temporarily interrupt provision of the physical quantity by intention of the user. In addition, it is also possible to use the sensor as a face sticker without providing the physical quantity in the first place.

(11) A provision method according to another embodiment of the present disclosure includes: a presentation step of presenting, to a user, a fact that contacting of a sensor with a body surface of the user serves as a condition for permission to provide a physical quantity to another entity other than the user, the sensor having a contact surface and being configured to measure the physical quantity, the physical quantity indicating a degree of change in a surface with which the contact surface contacts; an estimation step of estimating a contact state of the sensor with the body surface on the basis of the physical quantity; and a provision step of starting providing the physical quantity to the other entity, on the basis of an estimation result for the contact state.

This configuration includes the steps corresponding to the features included in the above provision system. Thus, it is possible to provide the same operations and effects as in the above provision system.

(12) A computer program according to another embodiment of the present disclosure causes a computer to function as: a presentation unit configured to present, to a user, a fact that contacting of a sensor with a body surface of the user serves as a condition for permission to provide a physical quantity to another entity other than the user, the sensor having a contact surface and being configured to measure the physical quantity, the physical quantity indicating a degree of change in a surface with which the contact surface contacts; an estimation unit configured to estimate a contact state of the sensor with the body surface on the basis of the physical quantity; and a provision unit configured to start providing the physical quantity to the other entity, on the basis of an estimation result for the contact state by the estimation unit.

With this configuration, it is possible to cause the computer to function as the above provision system. Thus, it is possible to provide the same operations and effects as in the above provision system.

### Details of Embodiment of the Present Disclosure

Hereinafter, an embodiment of the present disclosure will be described with reference to the drawings. The embodiment described below merely shows, in any case, a specific example of the present disclosure. Numerical values, shapes, materials, components, arrangement positions and connection manners of components, steps, the order of steps, and the like are merely examples, and are not intended to limit the present disclosure. Among the components in the embodiment below, a component not described in the independent claims is a component that can be arbitrarily added. The drawings are given schematically and do not necessarily provide strict illustrations.

The same components are denoted by the same reference signs. The same applies to functions and names thereof and therefore the description thereof is omitted as appropriate.

### [Entire Configuration of Emotion Analysis System]

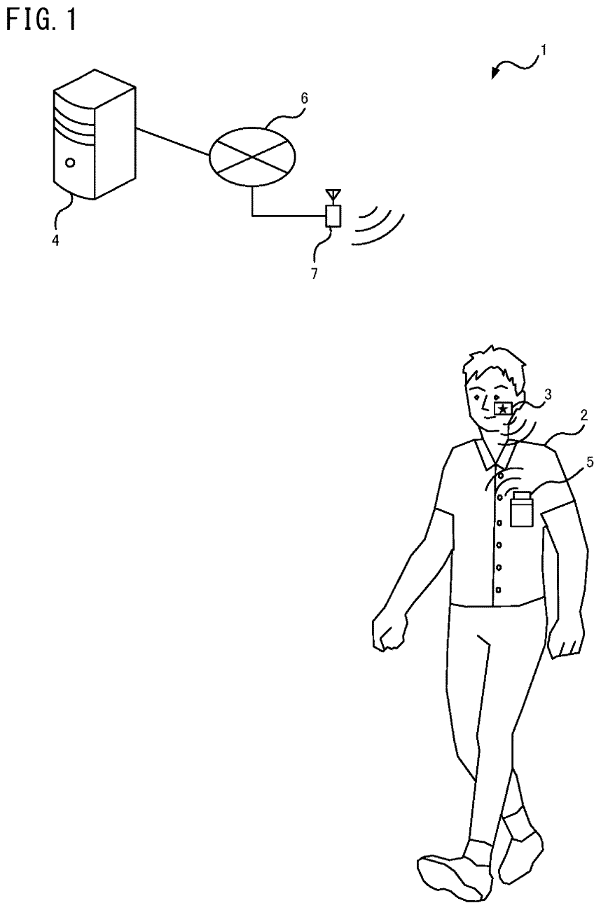

FIG. 1 shows an example of an entire configuration of an emotion analysis system according to an embodiment.

An emotion analysis system 1 includes a sensor 3, a server 4, and a smartphone 5.

The sensor 3 has an adhesion surface and has a sheet shape. The sensor 3 is pasted on the face of a user 2 (human) via the adhesion surface, for example, and measures a physical quantity indicating the degree of change in the shape of the face according to change in the emotion. The adhesion surface of the sensor 3 is an example of a contact surface of the sensor 3 with the face. That is, the sensor 3 can measure the physical quantity also by contacting with the face using fixation means or the like, without adhering the sensor 3 to the face. The sensor 3 may be used by being pasted on a body surface of the user 2 other than the face. The sheet shape which is the shape of the sensor 3 may be a shape having no recess/projection on a surface, or may be a spatial shape having a slight recess/projection on a surface of the sensor 3 from the perspective of fashion, function, or the like. For example, from the perspective of fashion, the sensor 3 may have decoration with a recess/projection on the surface thereof, or the surface of the sensor 3 may have a recess/projection shape. From the perspective of function, the contact surface of the sensor 3 may have a recess/projection to ease contact with skin.

In FIG. 1, only the sensor 3 pasted on one user 2 is shown, but such a sensor 3 is pasted on each of a plurality of users 2.

The sensor 3 is capable of near field wireless communication with the smartphone 5. For example, the sensor 3 performs BLE (Bluetooth (registered trademark) Low Energy) communication with the smartphone 5. The sensor 3 has a BLE beacon function and outputs radio waves in a 2.4 GHz band, for example, to broadcast a measured physical quantity and a sensor ID (sensor identifier) for identifying the sensor 3 relative to the other sensors 3, in real time. The BLE communication uses adaptive frequency hopping, and thus can reduce interference between radio waves transmitted from the sensor 3 and radio waves transmitted from another sensor 3.

The smartphone 5 is a terminal device that the user 2 has, and is capable of near field wireless communication with the sensor 3. The smartphone 5 has a receiver function of receiving the physical quantity and the sensor ID transmitted from the sensor 3 by performing BLE communication with the sensor 3, for example.

The smartphone 5 is connected to a network 6 such as the Internet via a wireless base station 7. Between the smartphone 5 and the wireless base station 7, wireless communication in conformity with a wireless communication standard such as a 5G (5th-generation mobile communication system) or Wi-Fi (registered trademark) is performed, for example.

The smartphone 5 extracts set data including the sensor ID registered in advance, from set data of the physical quantities and the sensor IDs from the sensors 3, and transmits set data obtained by adding position information of the smartphone 5 to the extracted set data, to the server 4 via the network 6 in real time. Since the user 2 has the smartphone 5, the server 4 can regard the position of the smartphone 5 as a measurement position for the physical quantity of the user 2.

The user 2 may use a terminal device capable of wireless communication, such as a mobile phone or a notebook computer, instead of the smartphone 5.

The server 4 is a device that is used by another entity other than the user 2. The other entity is a business entity that analyzes the emotion of the user 2 by using data collected from the user 2, for example. The server 4 functions as an emotion analysis device, is connected to the network 6 via a wire or wirelessly, and receives set data including the physical quantity, the sensor ID, and the position information from each of a plurality of smartphones 5 via the network 6. Since the sensor 3 and the smartphone 5 transmit the set data in real time, the server 4 can estimate the time when the set data is received, as a measurement time for the physical quantity.

In the server 4, for each user 2, user information including personal information such as the gender and the birth date of the user 2, and the sensor ID of the sensor 3 used by the user 2, is registered in advance. Therefore, the server 4 can specify the user 2 corresponding to the received set data on the basis of the received set data and user information, and analyzes, for each user 2, the emotion of the user 2 on the basis of the physical quantity included in the set data.

For example, at the time of entering an event venue such as a sports watching venue or an attraction venue, an event staff member hands out the sensor 3 to the user 2 who is a participant. The user 2 who has received the sensor 3 pastes the sensor 3 on the own face (e.g., a cheek part), thereby bringing the sensor 3 into contact with the face. In order for the user 2 to paste the sensor 3 without resistance, it is desirable that a letter, an image (e.g., a mark of a team for which the user 2 cheers), or the like is printed on a surface of the sensor 3 that can be seen from others. Thus, the user 2 can paste the sensor 3 on the face with a feeling as if pasting a face sticker.

The server 4 can analyze the emotion of the user 2 for each position or each area on the basis of position information included in the set data received from the smartphone 5. For example, the server 4 analyzes the emotion of the user for each attraction (e.g., a roller coaster or a merry-go-round) in an attraction venue (e.g., a theme park) or the like. Thus, the server 4 can analyze a popular attraction, for example.

### [Configurations of Sensor 3 and Server 4]

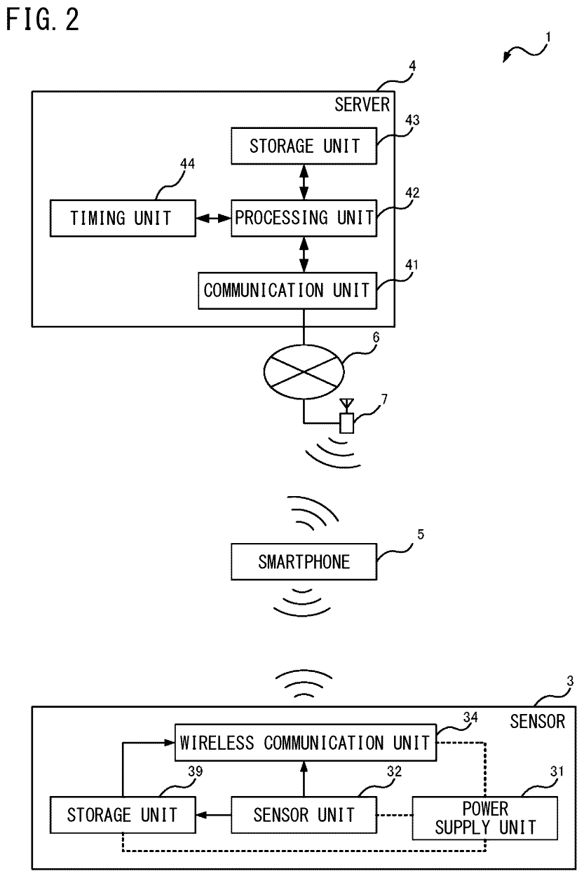

FIG. 2 is a block diagram showing an example of a configuration of each of the sensor 3 and the server 4 constituting the emotion analysis system 1 according to the embodiment.

The sensor 3 includes a power supply unit 31, a sensor unit 32, a wireless communication unit 34, and a storage unit 39.

The power supply unit 31 supplies the sensor unit 32, the wireless communication unit 34, and the storage unit 39 with power for driving each processing unit. The power supply unit 31 is, for example, a sheet battery or a patch battery (see, for example, NON PATENT LITERATURE 2). In FIG. 2, supply lines for power are indicated by broken lines.

The power supply unit 31 may generate power using energy of a living body and supply the generated power. For example, the power supply unit 31 performs temperature difference power generation using thermal energy of the user 2 on which the sensor 3 is pasted. More specifically, the power supply unit 31 includes a sheet-shaped Seebeck element (thermoelectric element) to contact with the skin of the user 2, and generates power using the Seebeck effect that an electromotive force is generated by a temperature difference inside the Seebeck element.

The power supply unit 31 may generate power using sweat of a living body. More specifically, the power supply unit 31 includes a sheet-shaped biofuel cell to contact with the skin of the user 2, and generates power by the biofuel cell converting human sweat into current by an enzyme that oxidizes lactic acid contained in the human sweat.

The power supply unit 31 may generate power using electromagnetic waves. More specifically, the power supply unit 31 may collect energy from electromagnetic waves present around the sensor 3 and generate power on its own (see, for example, NON PATENT LITERATURE 3).

The power supply unit 31 is not limited to the above examples.

The storage unit 39 is a storage device for storing the sensor ID of the sensor 3.

The sensor unit 32 measures a physical quantity indicating the degree of change in the shape of the surface (the face surface of the user 2) with which the adhesion surface of the sensor unit 32 contacts. The sensor unit 32 includes a strain gauge, for example. The strain gauge measures a force applied to the strain gauge by movement of the skin of the face of the user 2, as current (hereinafter, referred to as “strain amount”) (physical quantity). The sensor unit 32 outputs the strain amount measured by the strain gauge, to the wireless communication unit 34.

The wireless communication unit 34 includes a small-sized and low-powered communication interface for performing wireless communication. The wireless communication unit 34 performs data communication in conformity with the communication standard of BLE as described above, for example. The wireless communication unit 34 may perform data communication in conformity with a communication standard such as Wi-SUN (Wireless Smart Utility Network) or ZigBee (registered trademark), for example. The wireless communication unit 34 receives the strain amount from the sensor unit 32, reads the sensor ID from the storage unit 39, and broadcasts the strain amount with the sensor ID added thereto, in real time.

The wireless communication unit 34 transmits set data of the strain amount measured by the sensor unit 32 and the sensor ID stored in the storage unit 39, to the server 4.

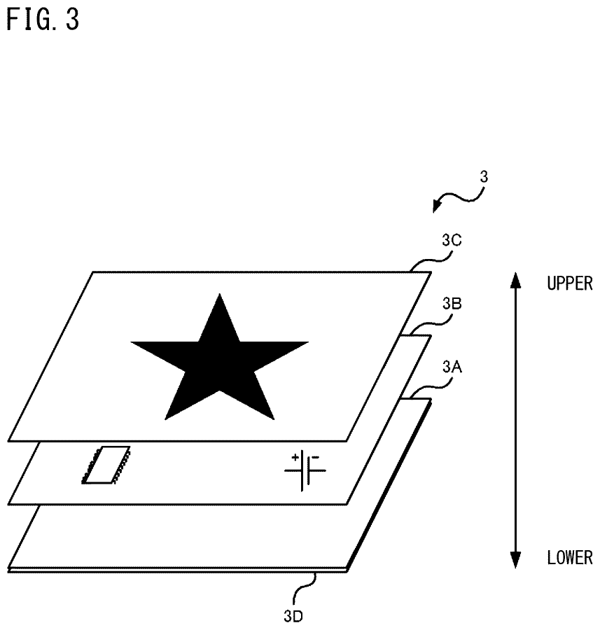

FIG. 3 shows an example of a structure of the sensor 3. In FIG. 3, in a case where the sensor 3 is pasted on the skin in normal usage, the side close to the skin is defined as a “lower” side and the side far from the skin is defined as an “upper” side. The sensor 3 is composed of three layers, i.e., a sheet-shaped adhesion layer 3A, a sheet-shaped circuit layer 3B, and a sheet-shaped print layer 3C, for example. A sticking material for skin, which is mild to skin, is applied on the lower side of the adhesion layer 3A. A peel-off sheet 3D is provided on the lower side of the sticking material. The user 2 peels the peel-off sheet 3D and then brings the lower side of the adhesion layer 3A into contact with the skin, thus pasting the sensor 3 on the skin. It is also possible that the user 2 peels the sensor 3 from the skin and then pastes the sensor 3 again.

The circuit layer 3B is located on the upper side of the adhesion layer 3A. At the circuit layer 3B, a circuit implementing the power supply unit 31, the sensor unit 32, the wireless communication unit 34, and the storage unit 39 described above is formed. The circuit layer 3B is formed by placing an IC (Integrated Circuit) chip on a flexible circuit board, for example. In a case where the power supply unit 31 performs temperature difference power generation or power generation using sweat, it is desirable that the Seebeck element or the biofuel cell contacts with the skin. Therefore, the adhesion layer 3A is not provided on the lower side of the Seebeck element or the biofuel cell, so that the Seebeck element or the biofuel cell contacts with the skin.

The print layer 3C is located on the upper side of the circuit layer 3B and has a print surface. The print surface is located on the upper side of the print layer 3C, and at least one of a letter or an image can be printed on the print surface. A part or the entirety of the print surface may be a blank surface with a white color or the like or may be transparent or translucent, so that the user 2 can draw a letter or an image on the print surface by a pen or the like.

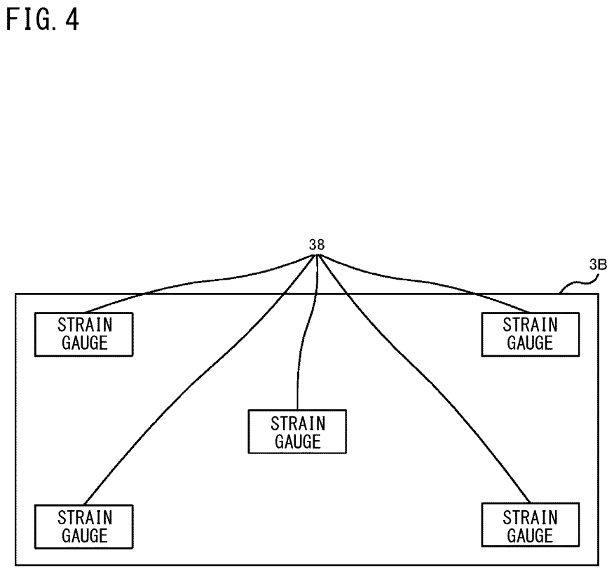

FIG. 4 shows an example of arrangement positions of strain gauges 38 at the circuit layer 3B. The strain gauges 38 constitute a part of the sensor unit 32. For example, the strain gauges 38 are located at four corners and a center part of the circuit layer 3B. By arranging the strain gauges 38 at a plurality of locations as described above, the sensor unit 32 can measure the degree of change in the shape of the face, with high accuracy as compared to a case of providing the strain gauge 38 at one location. Here, the strain amount measured by the sensor unit 32 is a representative value (e.g., an average value, a maximum value, a minimum value, or a median value) of the strain amounts measured by the plurality of strain gauges 38. Alternatively, a set of the strain amounts measured by the plurality of strain gauges 38 may be used as the strain amount measured by the sensor unit 32.

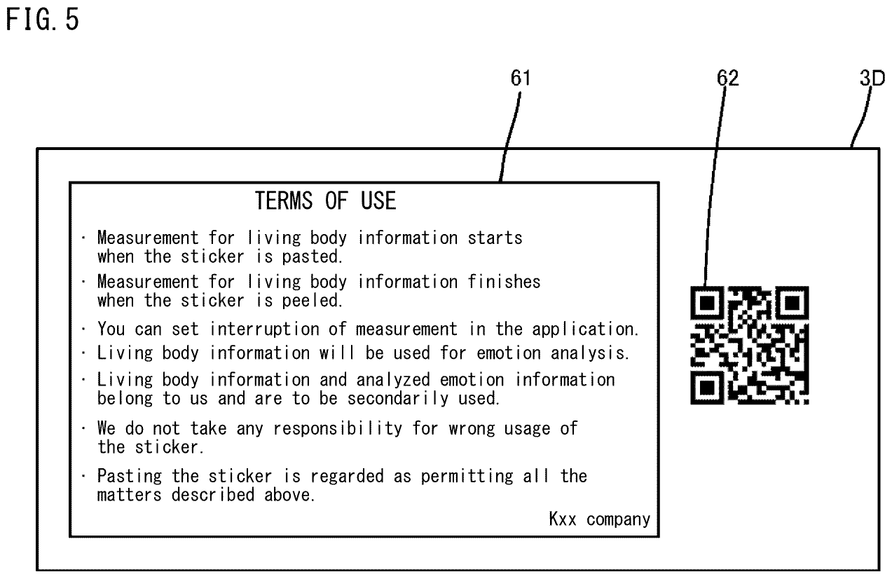

FIG. 5 shows an example of the peel-off sheet 3D composing the sensor 3. Terms of use 61 and a QR code (registered trademark) 62 are printed in advance on a lower surface (i.e., a surface that the user 2 can see without peeling the peel-off sheet 3D) of the peel-off sheet 3D.

The terms of use 61 indicate terms for the user 2 to use the sensor 3. The terms of use 61 indicate, in particular, that pasting the sensor 3 serves as a condition for permission to provide the strain amount to another entity other than the user 2. Specifically, the terms of use 61 indicate that measurement for living body information (strain amount) starts when the sticker (sensor 3) is pasted and that the living body information is used for emotion analysis. In addition, the terms of use 61 may indicate that the living body information and analyzed emotion information belong to a corresponding company (here, Kxx company which uses the server 4) and are to be secondarily used. Besides, the terms of use 61 indicate that measurement for living body information is finished when the sticker is peeled from the body surface, that interruption of measurement can be set in an application, that the company does not take any responsibility for wrong usage of the sticker, and that pasting the sticker is regarded as permitting all the matters described, for example.

The QR code 62 includes information obtained by encoding the sensor ID of the sensor 3. By the user 2 reading the QR code 62 using a camera of the smartphone 5, the smartphone 5 can acquire the sensor ID of the sensor 3.

With reference to FIG. 2 again, the server 4 includes a communication unit 41, a processing unit 42, a storage unit 43, and a timing unit 44.

The server 4 can be formed by a computer including a CPU (Central Processing Unit), a ROM (Read Only Memory), a RAM (Random Access Memory), an HDD (Hard Disk Drive), a communication interface, and the like. The processing unit 42 is functionally implemented by loading a computer program stored in a nonvolatile memory such as the ROM or the HDD onto the RAM and executing the computer program on the CPU.

The communication unit 41 receives the set data of the strain amount, the sensor ID, and the position information from the smartphone 5 via the network 6.

The timing unit 44 is a clock or a timer for measuring the time.

The storage unit 43 stores, for each user 2, user information including the user ID (user identifier) for identifying the user 2, personal information such as the gender and the birth date of the user 2, and the sensor ID of the sensor 3 used by the user 2.

The processing unit 42 stores, in the storage unit 43, the set data received by the communication unit 41 and the time measured by the timing unit 44, with the time included in the set data. The time measured by the timing unit 44 is estimated as a measurement time for the strain amount. In addition, the processing unit 42 reads the set data and the user information from the storage unit 43. On the basis of the read set data and user information, the processing unit 42 specifies the user 2 who uses the set data. That is, the processing unit 42 specifies the sensor ID included in the set data, and specifies the user ID corresponding to the specified sensor ID, on the basis of the user information.

The processing unit 42 analyzes an emotion for each user ID (for each user 2), on the basis of the strain amount included in the set data corresponding to the user ID. The emotion to be analyzed is, for example, joy of the user 2. For example, using a trained model that has learned in advance the relationship between time-series strain amounts and joy levels which are numerical values indicating the degree of joy, the processing unit 42 determines a joy level by inputting the measurement time and the strain amount to the trained model. The trained model is, for example, a multilayer neural network, and undergoes machine learning for parameters of the neural network by deep learning using a training set with the measurement time and the strain amount as an input and the degree of joy as an output. The trained model is not limited to the neural network. Another discriminator such as linear regression model, logistic regression model, support vector machine, random forest, Ada Boost, naive Bayes, or k-nearest neighbors algorithm may be used, for example.

The processing unit 42 may further create display data for displaying an analysis result of the joy level for each user 2 on a screen of a display device. The display data may indicate distribution of the joy levels of a plurality of users 2 in a table format or a graph format, or may indicate the joy level in association with the position of the user 2 in a map format, for example.

The user ID is associated with the personal information. Therefore, the processing unit 42 may analyze the emotion of the user 2 on a personal information basis. For example, the processing unit 42 may analyze the emotion of the user 2 on a gender basis or may analyze the emotion of the user 2 on an age basis.

The set data received by the communication unit 41 includes the position information. Therefore, the processing unit 42 may analyze the emotion of the user 2 for each position or each area.

### [Configuration of Smartphone 5]

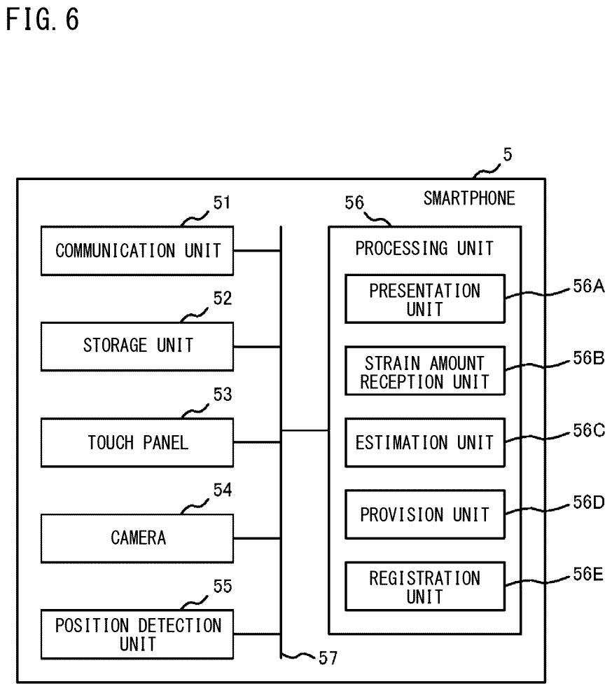

FIG. 6 is a block diagram showing an example of a configuration of the smartphone 5 according to the embodiment.

The smartphone 5 includes a communication unit 51, a storage unit 52, a touch panel 53, a camera 54, a position detection unit 55, and a processing unit 56 which are connected to each other via an internal bus 57.

The communication unit 51 includes a small-sized and low-powered communication interface for performing wireless communication. The wireless communication unit 34 receives the set data broadcast from the sensor 3 in conformity with the communication standard of BLE as described above, for example. The communication unit 51 may perform data communication in conformity with a communication standard such as Wi-SUN or ZigBee (registered trademark), for example.

The communication unit 51 is connected to the network 6 via the wireless base station 7 through communication in conformity with a wireless communication standard such as 5G or Wi-Fi (registered trademark).

The storage unit 52 stores the sensor ID of the sensor 3 to be pasted on the user 2 who has the smartphone 5. There may be a user 2 who does not have the smartphone 5. Therefore, the storage unit 52 may store the sensor ID of the sensor 3 to be pasted on a user 2 other than the user 2 who has the smartphone 5. Thus, for example, the storage unit 52 stores the sensor IDs of the sensors 3 respectively pasted on the user 2 who has the smartphone 5 and the other user 2 accompanying the user 2.

The storage unit 52 is formed by a nonvolatile memory or a volatile memory, for example.

The touch panel 53 has functions as a display device (display panel) which displays various information to the user 2, and an input device (touch sensor) which receives an input of various information from the user 2.

The camera 54 is used for reading the QR code 62 printed on the peel-off sheet 3D of the sensor 3 shown in FIG. 5.

The position detection unit 55 detects the position of the smartphone 5, using satellite navigation. For example, the position detection unit 55 detects the position of the smartphone 5 on the basis of radio waves received from a plurality of GPS (Global Positioning System) satellites. The position of the smartphone 5 can be specified by a latitude and a longitude, for example. The satellite navigation is performed using a satellite positioning system (GNSS: Global Navigation Satellite System) such as GPS, but is not limited to the GPS.

The processing unit 56 is formed by a processor such as a CPU, for example.

The processing unit 56 includes a presentation unit 56A, a strain amount reception unit 56B, an estimation unit 56C, a provision unit 56D, and a registration unit 56E, as functional processing units implemented through execution of a computer program stored in advance in the storage unit 52.

The presentation unit 56A displays terms of use for using the sensor 3, on the touch panel 53. The terms of use displayed on the touch panel 53 includes contents similar to the terms of use 61 printed on the peel-off sheet 3D of the sensor 3 shown in FIG. 5. That is, the terms of use displayed on the touch panel 53 indicate, in particular, that pasting the sensor 3 serves as a condition for permission to provide the strain amount to another entity other than the user 2.

The strain amount reception unit 56B receives the set data of the strain amount and the sensor ID of the sensor 3, from the sensor 3 via the communication unit 51. The estimation unit 56C estimates the contact state of the sensor 3 with the body surface of the user 2, on the basis of the strain amount included in the set data received by the strain amount reception unit 56B. That is, the estimation unit 56C estimates that the sensor 3 is in contact with the body surface of the user 2 or that the sensor 3 is not in contact with the body surface of the user 2, on the basis of the strain amount. The estimation method for the contact state will be described later.

The provision unit 56D starts to provide the strain amount to the server 4, on the basis of an estimation result for the contact state by the processing unit 56. That is, when the estimation unit 56C has estimated that the sensor 3 is in contact with the body surface of the user 2, the provision unit 56D starts to provide the set data including the strain amount, to the server 4.

Specifically, the provision unit 56D adds the position information detected by the position detection unit 55, to the set data including the sensor ID stored in the storage unit 52 among the set data received by the strain amount reception unit 56B, and provides the resultant set data to the server 4 via the communication unit 51.

The provision unit 56D discards the set data that does not include the sensor ID stored in the storage unit 52 among the set data received by the strain amount reception unit 56B, and does not provide the discarded set data to the server 4.

When the estimation unit 56C has estimated that the sensor 3 is not in contact with the body surface of the user 2, the provision unit 56D finishes providing the strain amount to the server 4.

The registration unit 56E registers the sensor ID of the sensor 3 to be pasted on the user 2. Specifically, the registration unit 56E stores, in the storage unit 52, the sensor ID acquired by reading the QR code 62 using the camera 54. In addition, the registration unit 56E transmits, to the server 4, the acquired sensor ID and the user ID of the user 2 as a set. The server 4 receives the set and registers the sensor ID and the user ID in association with each other, as user information.

The registration unit 56E executes registration processing for an event in which the user 2 participates.

### [User Registration Processing]

Hereinafter, each processing executed by the emotion analysis system 1 will be described.

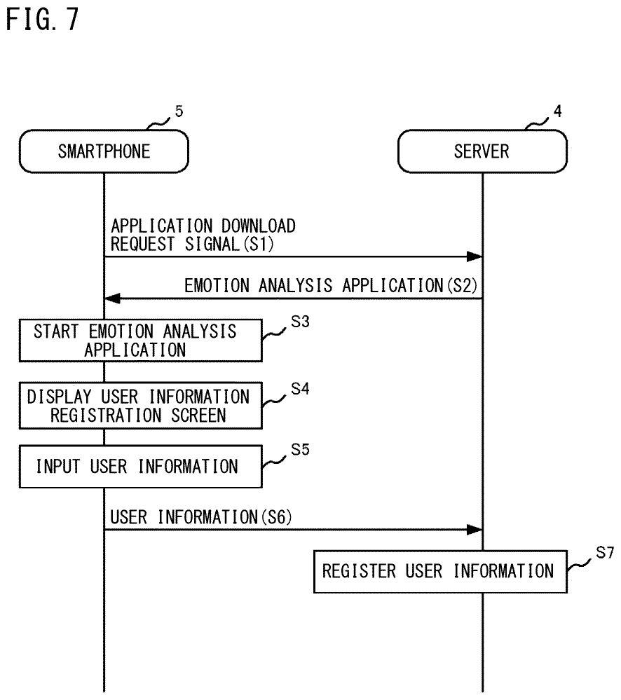

FIG. 7 is a sequence diagram showing an example of user registration processing for registering the user information of the user 2 who uses the sensor 3, in the server 4.

The user 2 operates the touch panel 53 of the smartphone 5, to make a request to the server 4 for download of an application of the emotion analysis system 1 (hereinafter, referred to as “emotion analysis application”). For example, the user 2 accesses a webpage of the server 4 where applications can be downloaded, and selects the emotion analysis application from the applications that can be downloaded. In response to the selection, the registration unit 56E transmits, to the server 4, a request signal for requesting download of the emotion analysis application. The processing unit 42 of the server 4 receives the request signal via the communication unit 41 (step S1).

The processing unit 42 of the server 4 reads the emotion analysis application from the storage unit 43, and transmits the read emotion analysis application to the smartphone 5 via the communication unit 41, and the registration unit 56E receives the emotion analysis application (step S2). The registration unit 56E installs the emotion analysis application on the smartphone 5. A source that provides the emotion analysis application is not limited to the server 4, and a dedicated server for providing applications, other than the server 4, may provide the emotion analysis application.

When the user 2 taps an icon of the emotion analysis application displayed on the touch panel 53, the processing unit 56 starts the emotion analysis application in response to the tap (step S3).

After the emotion analysis application is started, when the user 2 selects a menu for user information registration, a user information registration screen is displayed (step S4).

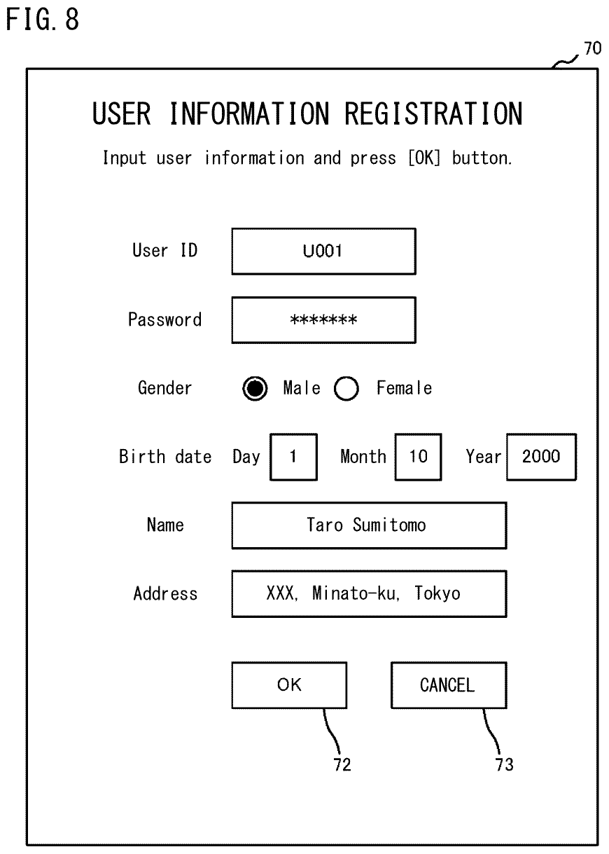

FIG. 8 shows an example of the user information registration screen. The user information registration screen includes input boxes for a user ID, a password, a gender, a birth date, a name, and an address, an OK button 72, and a cancel button 73. For example, the user 2 operates the touch panel 53 to input user information in each input box, and then taps the OK button 72 (step S5). The registration unit 56E transmits the inputted user information to the server 4 via the communication unit 51, and the processing unit 42 of the server 4 receives the user information from the smartphone 5 via the communication unit 41 (step S6). The processing unit 42 stores the received user information in the storage unit 43 (step S7).

The user 2 can also cancel the user information registration processing by tapping the cancel button 73. In addition, the user 2 can also register user information of a plurality of users 2. For example, the user 2 registers user information of another user 2 accompanying the user 2.

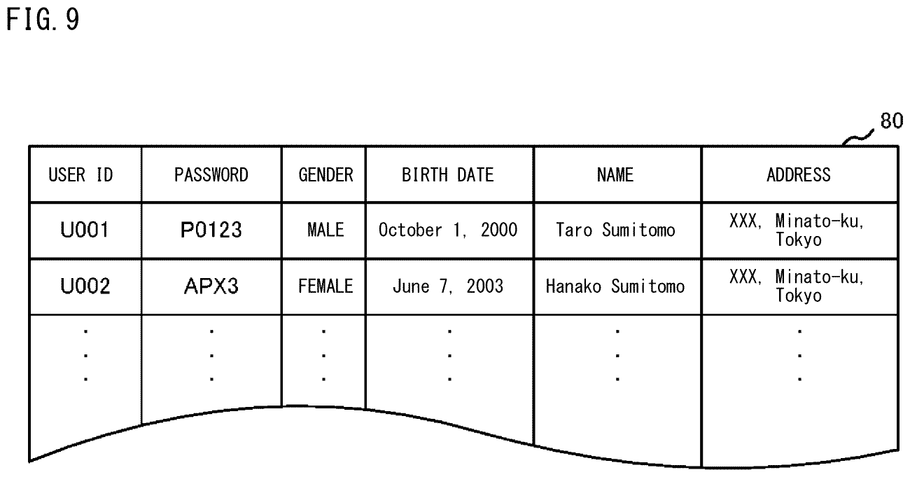

FIG. 9 shows an example of the user information registered in the server 4. User information 80 includes a user ID, a password, a gender, a birth date, a name, and an address. For example, for the user 2 whose user ID is “U001”, the password is “P0123”, the gender is “male”, the birth date is “Oct. 1, 2000”, the name is “Taro Sumitomo”, and the address is “XXX, Minato-ku, Tokyo”. For the user 2 whose user ID is “U002”, the password is “APX3”, the gender is “female”, the birth date is “Jun. 7, 2003”, the name is “Hanako Sumitomo”, and the address is “XXX, Minato-ku, Tokyo”. Here, the user 2 whose user ID is “U002” is a person accompanying the user 2 whose user ID is “U001”.

### [Event Registration Processing]

After the user registration processing is completed, the user 2 executes registration processing for an event in which the user 2 and the accompanying person participate.

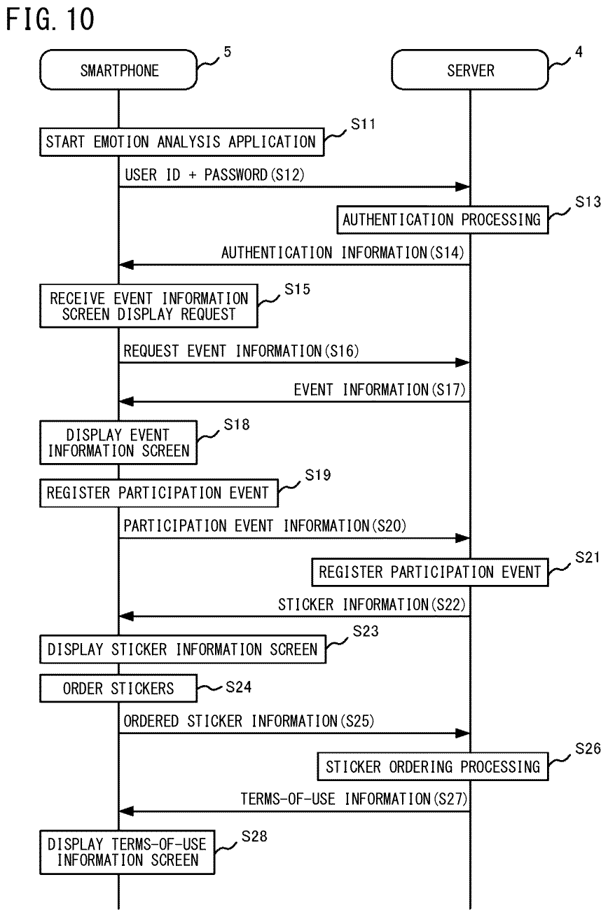

FIG. 10 is a sequence diagram showing an example of the event registration processing.

When the user 2 taps the icon of the emotion analysis application displayed on the touch panel 53 of the smartphone 5, the processing unit 56 starts the emotion analysis application in response to the tap (step S11).

After the application is started, when the user 2 operates the touch panel 53 to input the user ID and the password, the processing unit 56 transmits the inputted user ID and password to the server 4, and the processing unit 42 of the server 4 receives the user ID and the password transmitted from the smartphone 5 (step S12).

The processing unit 42 executes authentication processing for determining whether or not the received user ID and password have been registered in the user information 80 (step S13).

If the user ID and the password have been registered, the processing unit 42 transmits authentication information indicating that authentication is done, to the smartphone 5 that is the transmission source, and the processing unit 56 of the smartphone 5 receives the information (step S14).

After authentication, when the user 2 selects a menu for event information screen display on the application (step S15), the registration unit 56E transmits a request signal for event information to the server 4, and in response to the request signal, the processing unit 42 reads information on events for which participants are being invited, from the storage unit 43, and transmits the event information to the smartphone 5. The registration unit 56E receives the event information (step S17).

On the basis of the received event information, the registration unit 56E displays an event information screen on the touch panel 53 (step S18).

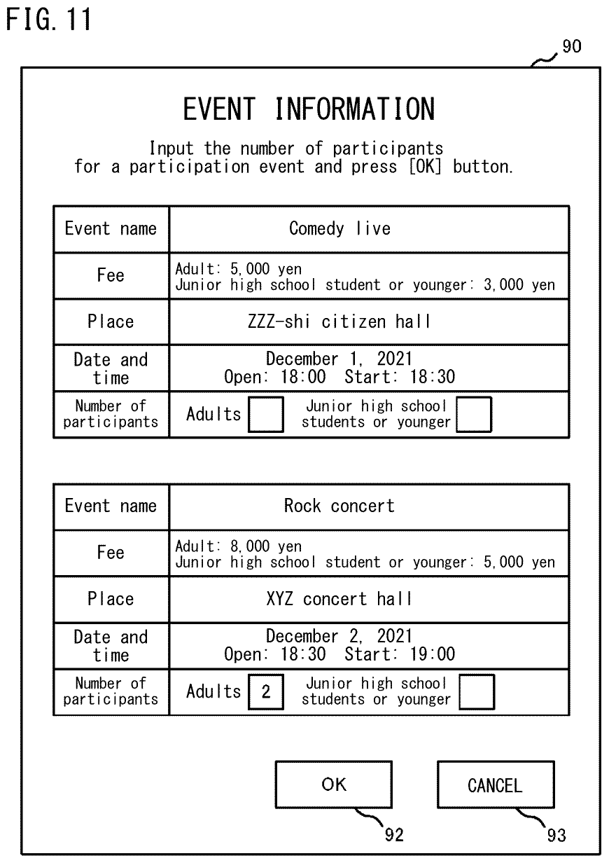

FIG. 11 shows an example of the event information screen. On an event information screen 90, as an example, event information is displayed for two events in which the user 2 can participate. However, the number of events is not limited to two.

Each event information includes an event name, a fee, a place, and a date and time. For example, for the first event, the event name is “Comedy live”, the fee is “Adult: 5,000 yen, Junior high school student or younger: 3,000 yen”, the place is “ZZZ-shi citizen hall”, and the date and time are “Dec. 1, 2021, Open: 18:00, Start: 18:30”. For the second event, the event name is “Rock concert”, the fee is “Adult: 8,000 yen, Junior high school student or younger: 5,000 yen”, the place is “XYZ concert hall”, and the date and time are “Dec. 2, 2021, Open: 18:30, Start: 19:00”.

On each event information, input boxes for the number of participants are provided. The user 2 inputs the number of participants for the event in which the user 2 hopes to participate, and taps the OK button 92, thereby making registration of the participation event (step S19). For example, the user 2 inputs “2” for “Adults” in a box for the number of participants for the event name “Rock concert”, and taps the OK button 92.

The registration unit 56E transmits participation event information including identification information (e.g., event name) on the event in which the user 2 hopes to participate and the number of participants, to the server 4, and the processing unit 42 receives the participation event information (step S20).

The processing unit 42 executes registration processing for the event in which the user 2 hopes to participate, on the basis of the participation event information (step S21). For example, information indicating that the user 2 participates as two adults in the “Rock concert” is registered in the storage unit 43.

On the event information screen 90, the user 2 can also cancel registration processing for the participation event by tapping the cancel button 93.

After the participation event registration processing (step S21), the processing unit 42 transmits sticker information indicating designs of the sensors 3 that can be handed out to the user 2 planning to participate, to the smartphone 5, and the registration unit 56E of the smartphone 5 receives the sticker information via the communication unit 51 (step S22).

The registration unit 56E displays a sticker information screen on the touch panel 53, on the basis of the received sticker information (step S23).

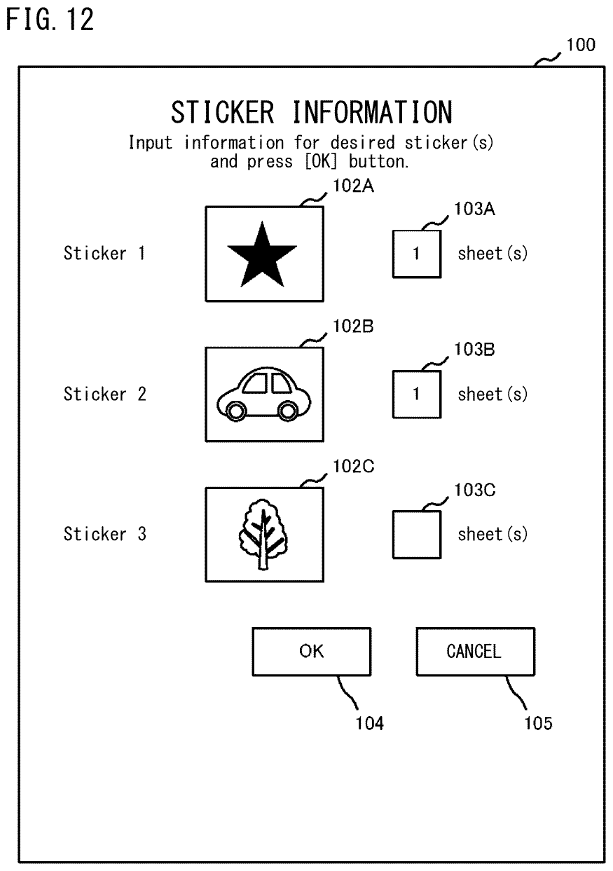

FIG. 12 shows an example of the sticker information screen. On a sticker information screen 100, three designs of stickers 102A to 102C are displayed as designs of the sensors 3 that can be handed out to the user 2. In addition, input boxes 103A to 103C for the numbers of sheets for the respective stickers 102A to 102C are displayed. The user 2 inputs the number of sheets for the desired sensor 3 in each of input boxes 103A to 103C for the numbers of sheets, and taps the OK button 104, thereby ordering a sticker(s) (step S24). For example, in a case of desiring one sheet for each of the sensors 3 with the sticker 102A and the sticker 102B, the user 2 inputs [1] in each of the input boxes 103A and 103B for the numbers of sheets, and taps the OK button 104. The user 2 can also stop ordering the sensor 3 by pressing the cancel button 105.

When the sticker ordering processing (step S24) is executed, the registration unit 56E transmits information on the ordered stickers to the server 4, and the processing unit 42 of the server 4 receives the information via the communication unit 41 (step S25). For example, identification information on the sticker 102B and the sticker 102C, and the numbers of ordered sheets (one for each), are transmitted to the server 4.

After receiving the information on the ordered stickers, the processing unit 42 executes sticker ordering processing (step S26). For example, the processing unit 42 transmits information such as the name and the address of the user 2, the designs and the numbers of sheets for the sensors 3 to be shipped, and identification information on the event for which the user 2 has made participation registration, to an email address of a person in charge for shipping the sensors 3. In a case of shipping the sensors 3 to the user 2, the person in charge for shipping ships the sensors 3 so that the sensors 3 will arrive at the user 2 by a day before the event is held. In a case of handing out the sensors 3 to the users 2 at an event venue, the person in charge for shipping ships the sensors 3 so that the sensors 3 will arrive at the event venue by a day before the event is held.

The processing unit 42 transmits terms-of-use information on the sensor 3 to the smartphone 5, and the presentation unit 56A of the smartphone 5 receives the terms-of-use information via the communication unit 51 (step S27).

The presentation unit 56A displays the terms-of-use information screen on the touch panel 53, on the basis of the received terms-of-use information (step S28).

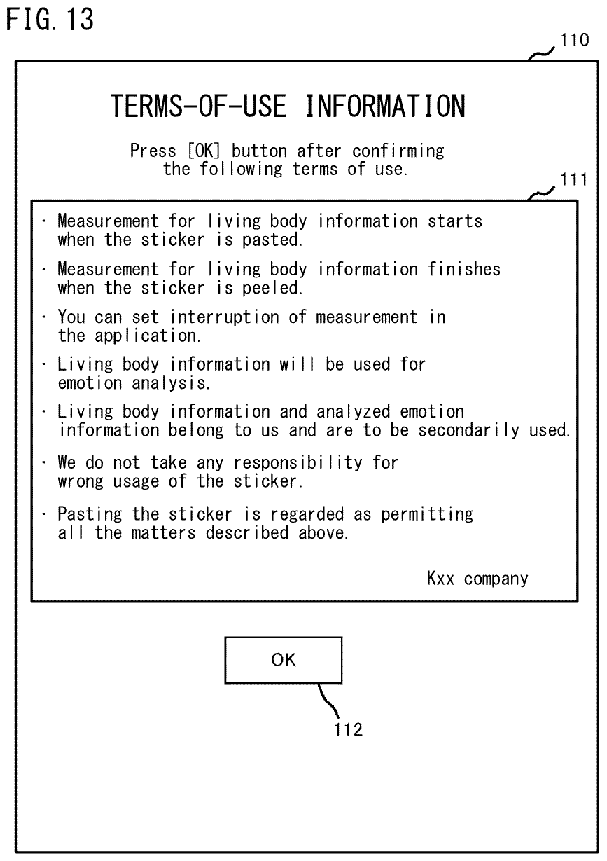

FIG. 13 shows an example of the terms-of-use information screen. On a terms-of-use information screen 110, terms of use 111 for the user 2 to use the sensor 3 and an OK button 112 for confirming that the user 2 agrees with the terms of use 111, are displayed. The terms of use 111 include contents similar to the terms of use 61 printed on the peel-off sheet 3D of the sensor 3 shown in FIG. 5.

The user 2 presses the OK button 112 after confirming the terms of use 111.

### [Linkage Processing]

After receiving the sensor 3, the user 2 executes linkage processing for linking the sensor ID of the sensor 3 with the user ID of the user 2 who uses the sensor 3.

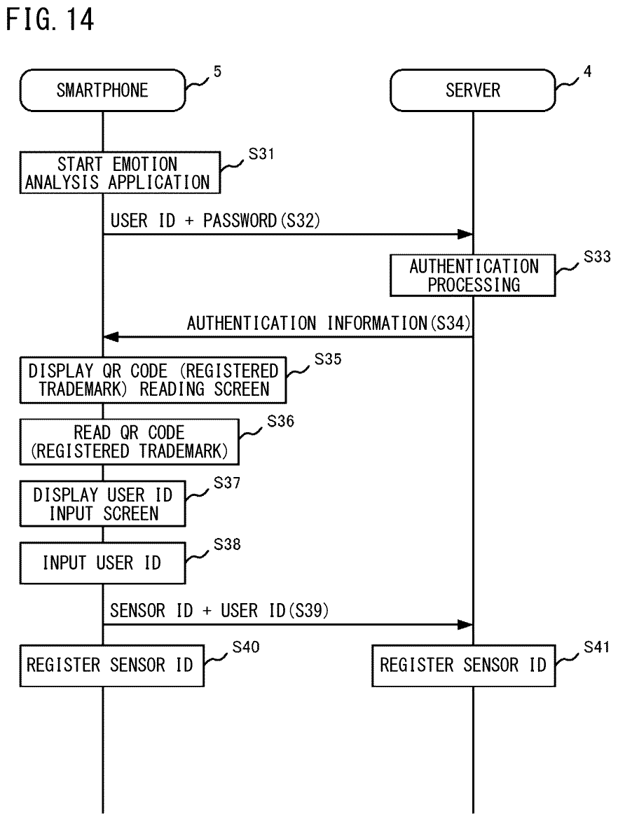

FIG. 14 is a sequence diagram showing an example of the linkage processing.

When the user 2 taps the icon of the emotion analysis application displayed on the touch panel 53 of the smartphone 5, the processing unit 56 starts the emotion analysis application in response to the tap (step S31).

After the application is started, when the user 2 operates the touch panel 53 to input the user ID and the password, the processing unit 56 transmits the inputted user ID and password to the server 4, and the processing unit 42 of the server 4 receives the user ID and the password transmitted from the smartphone 5 via the communication unit 41 (step S32).

The processing unit 42 executes authentication processing for determining whether or not the received user ID and password have been registered in the user information 80 (step S33).

If the user ID and the password have been registered, the processing unit 42 transmits authentication information indicating that authentication is done, to the smartphone 5 that is the transmission source, and the processing unit 56 of the smartphone 5 receives the information via the communication unit 51 (step S34).

After authentication, when the user 2 selects a menu for linkage processing on the application, the registration unit 56E displays, on the touch panel 53, a QR code reading screen for reading the QR code 62 printed on the sensor 3 (step S35).

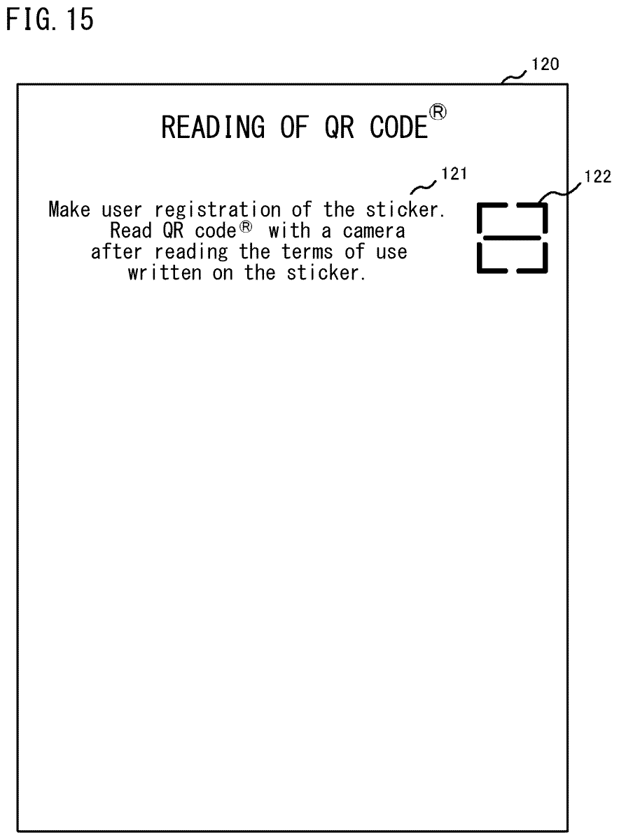

FIG. 15 shows an example of a QR code (registered trademark) reading screen. On a QR code reading screen 120, an explanatory text 121 about reading of the QR code 62, and an icon 122, are displayed. When the user 2 taps the icon 122, the registration unit 56E starts the camera 54. When the user 2 uses the camera 54 to capture the QR code 62 (FIG. 5) printed on the peel-off sheet 3D of the sensor 3, the registration unit 56E decodes the captured QR code 62 and acquires the sensor ID of the sensor 3 (step S36).

After acquiring the sensor ID, the processing unit 56 displays a user ID input screen for inputting the user ID to be linked with the sensor ID, on the touch panel 53 (step S37).

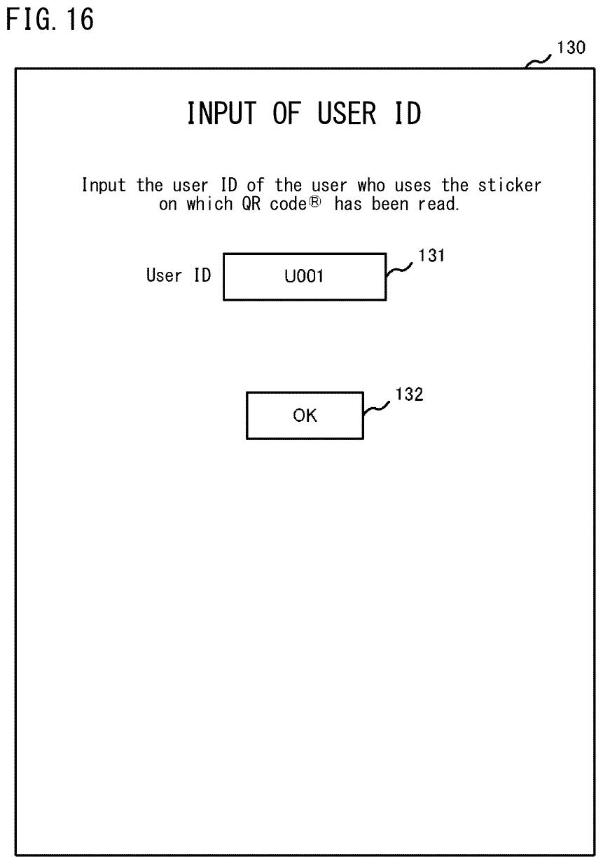

FIG. 16 shows an example of the user ID input screen. On a user ID input screen 130, an input box 131 for the user ID to be associated with the sensor ID acquired by the registration unit 56E, and an OK button 132 for deciding the input of the user ID, are displayed.

When the user 2 inputs the user ID to the input box 131 and taps the OK button 132 (step S38), the registration unit 56E transmits the acquired sensor ID and the inputted user ID as a set to the server 4. The processing unit 42 of the server 4 receives the set of the sensor ID and the user ID via the communication unit 41 (step S39).

On the basis of the received set, the processing unit 42 additionally registers the sensor ID in the user information 80 (step S41).

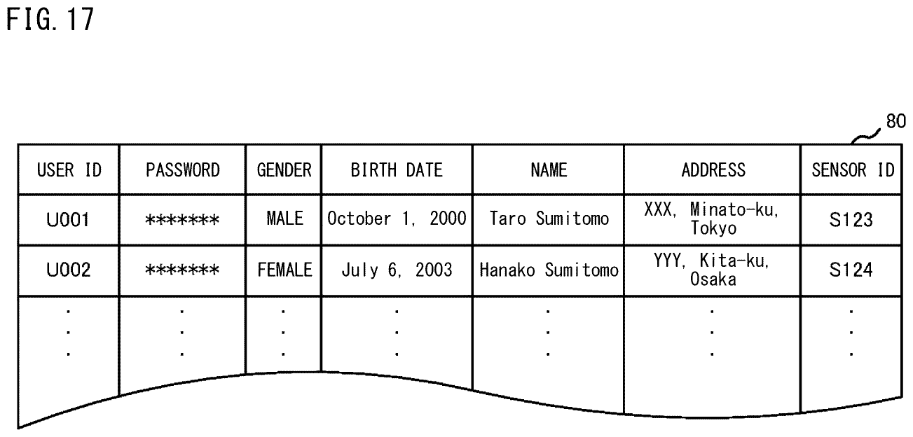

FIG. 17 shows an example of the user information in which the sensor ID is additionally registered. In the user information 80, boxes for sensor IDs are added. For example, in a case where S123 and U001 are received as a set of the sensor ID and the user ID, the processing unit 42 additionally registers the sensor ID “S123” in association with the user ID “U001” in the user information 80.

The registration unit 56E stores, in the storage unit 52, at least the sensor ID, of the sensor ID and the user ID transmitted to the server 4 in step S39, thereby making registration in the smartphone 5 (step S40). In the above example, the sensor ID “S123” is registered in the smartphone 5.

By repeatedly executing processing in step S35 and the subsequent steps, it is possible to register a plurality of sensor IDs and a plurality of user IDs in association with each other. For example, in the user information 80 shown in FIG. 17, a sensor ID “S124” is also additionally registered in association with the user ID “U002” in the user information 80. In addition, the sensor ID “S124” is also registered in the smartphone 5.

### [Strain Amount Measurement Processing]

After the linkage processing for the sensor ID and the user ID, the user 2 can start to use the sensor 3.

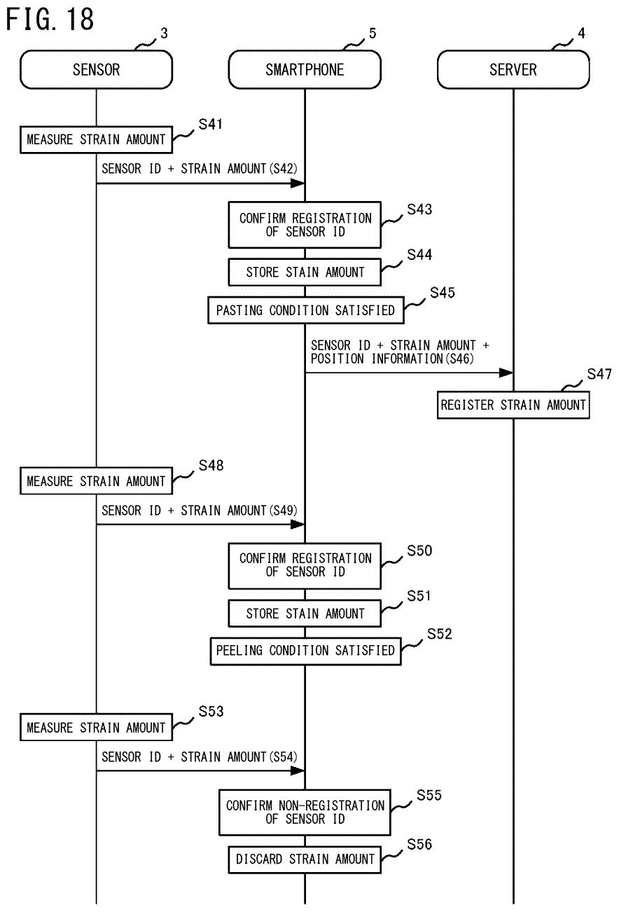

FIG. 18 is a sequence diagram showing an example of strain amount measurement processing.

When the user 2 peels the peel-off sheet 3D of the sensor 3 or turns on a power switch provided to the sensor 3, supply of power to each processing unit from the power supply unit 31 of the sensor 3 is started and the sensor unit 32 measures the strain amount of the strain gauge 38 (step S41).

The wireless communication unit 34 of the sensor 3 transmits set data including the measured strain amount and the sensor ID of the sensor 3 stored in the storage unit 39, to the smartphone 5, and the strain amount reception unit 56B of the smartphone 5 receives the set data (step S42).

The strain amount reception unit 56B determines whether or not the sensor ID included in the received set data has already been registered in the storage unit 52, and here, it is assumed that the strain amount reception unit 56B has confirmed that the sensor ID has already been registered (step S43).

The strain amount reception unit 56B stores, in the storage unit 52, the set data including the already registered sensor ID in association with the reception time for the set data (step S44).

On the basis of the set data and the reception time stored in the storage unit 52, the estimation unit 56C estimates, for each sensor ID, the contact state of the sensor 3 with the body surface of the user 2 from the strain amount and the reception time associated with the sensor ID. Here, it is assumed that a contact condition indicating that the sensor 3 is in contact with the body surface is satisfied (step S45).

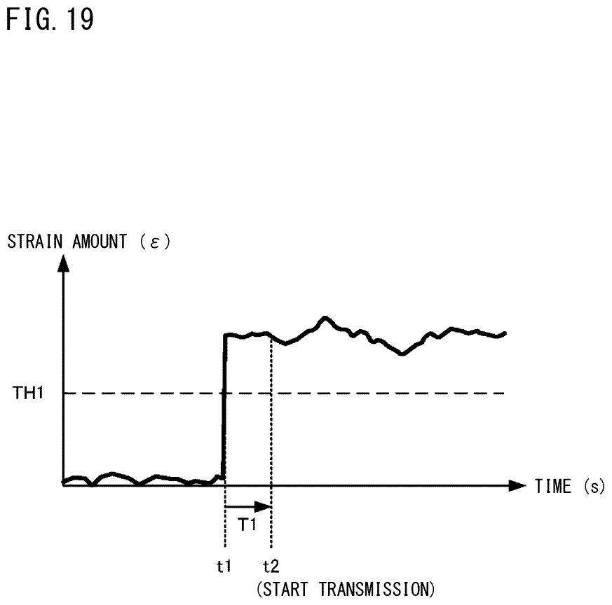

FIG. 19 shows an example of temporal change in the strain amount. The horizontal axis indicates time(s), and the vertical axis indicates a strain amount (ε). For example, the estimation unit 56C determines that the above contact condition is satisfied at a time (measurement time t2) when a period in which the strain amount is a predetermined threshold TH1 or greater has exceeded a predetermined period T1 starting from a time (measurement time t1) when the strain amount has become the predetermined threshold TH1 or greater. The estimation unit 56C may determine that the above contact condition is satisfied at the time (measurement time t1) when the strain amount has become the predetermined threshold TH1 or greater.

If the contact condition is satisfied, the provision unit 56D adds the position information detected by the position detection unit 55 to the set data of the sensor ID and the strain amount received in step S42, and transmits the resultant set data to the server 4. The processing unit 42 of the server 4 receives the set data with the position information added thereto, via the communication unit 41 (step S46).

The processing unit 42 adds the present time measured by the timing unit 44 to the received set data, and stores the resultant set data in the storage unit 43 (step S47). That is, the set data including the sensor ID, the strain amount, the position information, and the time information is registered in the storage unit 43.

If the contact condition is not satisfied and a non-contact condition indicating that the sensor 3 is not in contact with the body surface is satisfied, processing in the following steps S48 to S52 is executed. That is, as in steps S41 to S44, the set data including the strain amount measured by the sensor 3 is stored in the storage unit 52 of the smartphone 5 (steps S48 to S51).

On the basis of the set data and the reception time stored in the storage unit 52, the estimation unit 56C estimates, for each sensor ID, the contact state of the sensor 3 with the body surface of the user 2 from the strain amount and the reception time associated with the sensor ID. Here, it is assumed that the non-contact condition indicating that the sensor 3 is not in contact with the body surface is satisfied (step S52).

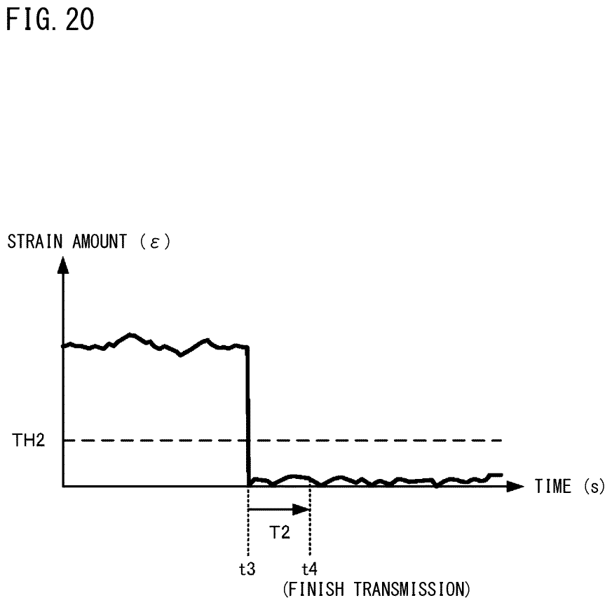

FIG. 20 shows an example of temporal change in the strain amount. The horizontal axis indicates time, and the vertical axis indicates the strain amount. For example, the estimation unit 56C determines that the above non-contact condition is satisfied at a time (measurement time t4) when a period in which the strain amount is smaller than a predetermined threshold TH2 has exceeded the predetermined period T2 starting from a time (measurement time t3) when the strain amount has become smaller than the predetermined threshold TH2. The estimation unit 56C may determine that the above non-contact condition is satisfied at the time (measurement time t3) when the strain amount has become smaller than the predetermined threshold TH2. Here, the threshold TH2 may be the same as or different from the threshold TH1. In addition, the predetermined period T2 may be the same as or different from the predetermined period T1.

If the non-contact condition is satisfied, the provision unit 56D does not transmit the set data of the sensor ID and the strain amount received in step S49, to the server 4.

For the set data including the sensor ID not registered in the smartphone 5, processing in the following steps S53 to S56 is executed. That is, as in steps S41 to S42, the smartphone 5 receives the set data of the sensor ID and the strain amount measured by the sensor 3, from the sensor 3 (steps S53 to S54).

The strain amount reception unit 56B determines whether or not the sensor ID included in the received set data has already been registered in the storage unit 52, and here, it is assumed that the strain amount reception unit 56B has confirmed that the sensor ID has not been registered (step S55).

The strain amount reception unit 56B discards the set data including the sensor ID that has not been registered (step S56). Thus, the provision unit 56D does not transmit the set data including the sensor ID that has not been registered, to the server 4.

### Effects of Embodiment

As described above, according to the embodiment of the present disclosure, the user 2 can paste the sensor 3 on a body surface such as the face after approving the condition that a strain amount obtained from the user 2 as a measurement target is provided to the server 4 when the sensor 3 is pasted on the body surface of the user 2. Thus, it is possible to prevent the strain amount of the user 2 from being provided to the server 4 while the user 2 does not know that fact. In addition, since the sensor 3 has a sheet shape, the user 2 can wear the sensor 3 with a feeling as if pasting a sticker on the face. In particular, at a sports watching venue, an attraction venue, or the like, the users 2 have a less feeling of resistance against pasting a sticker on the face. Therefore, the server 4 can collect strain amounts without giving a feeling of strangeness and a feeling of resistance to the users 2 and thus can analyze the emotions of the users 2.

When the sensor 3 is not in contact with the body surface of the user 2, change in the contact surface is small and the strain amount is also small, but for example, when the sensor 3 is pasted on the face surface of the user 2, the strain amount increases with movement of the mimic muscle or the like. Accordingly, when the strain amount is the threshold TH1 or greater, the estimation unit 56C can estimate that the sensor 3 is in contact with the body surface, and when it is estimated that the sensor 3 is in contact with the body surface, the provision unit 56D can start to provide the strain amount to the server 4. Thus, it can be estimated that the sensor 3 is in contact with the body surface and it is possible to prevent the strain amount from being provided to the server 4 before the sensor 3 contacts with the body surface. Therefore, the server 4 can analyze the emotion accurately.

In a case where the sticker is temporarily deformed due to external pressure or the like before contacting with the body surface, the strain amount temporarily becomes great, but the state in which the strain amount is great does not continue. Therefore, when the state in which the strain amount is the threshold TH1 or greater has continued during the predetermined period T1, the estimation unit 56C can estimate that the sensor 3 is in contact with the body surface.

When the sensor 3 is not in contact with the body surface, change in the contact surface is small and the strain amount is also small. Accordingly, for example, when the strain amount has become smaller than the threshold TH2, the estimation unit 56C can estimate that the sensor 3 is not in contact with the body surface, and when it is estimated that the sensor 3 is not in contact with the body surface, the provision unit 56D can finish providing the strain amount to the server 4. Thus, it can be estimated that the sensor 3 is not in contact with the body surface and it is possible to prevent the strain amount from being provided to the server 4 while the sensor 3 is not in contact with the body surface. Therefore, the server 4 can analyze the emotion accurately.

Even in a state in which the sensor 3 is in contact with the body surface, in such a case where, for example, the user's face temporarily becomes expressionless, the physical quantity temporarily becomes small, but the body surface slightly vibrates. Thus, even under the expressionless face, certain strain arises and the state in which the strain amount is small does not continue. Therefore, for example, when the state in which the strain amount is smaller than the threshold TH2 has continued during the predetermined period T2, it can be estimated that the sensor 3 is not in contact with the body surface.

The registration unit 56E of the smartphone 5 registers the sensor ID of the sensor 3 used by the user 2 or the accompanying person. The strain amount reception unit 56B receives the set data of the strain amounts and the sensor IDs from a plurality of sensors 3. The provision unit 56D provides, to the server 4, the set data including the sensor ID registered by the registration unit 56E among the set data received by the strain amount reception unit 56B. Thus, it is possible to efficiently provide the strain amounts of the user 2 and the accompanying person to the server 4.

The provision unit 56D does not provide, to the server 4, the set data including the sensor ID not registered in the smartphone 5 among the set data received by the strain amount reception unit 56B. For example, if the smartphone 5 in which the sensor ID is registered is uniquely set, the set data of this sensor ID and the corresponding strain amount is provided to the server 4 from only one smartphone 5. Thus, it is possible to prevent duplicate strain amounts from being provided to the server 4.

In the server 4, the user information 80 including the user IDs and the sensor IDs is registered. Thus, the server 4 can specify the user 2 from the set data of the strain amount and the sensor ID provided from the smartphone 5, and can analyze the emotion for each user 2. It is noted that the user ID is not included in the set data provided from the smartphone 5 to the server 4. Therefore, even if a third party intercepts the set data, the third party cannot specify the user 2 for which the strain amount has been measured. Thus, the privacy of the user 2 is protected.

The provision unit 56D of the smartphone 5 transmits the set data with the measurement position information for the strain amount included therein, to the server 4. Thus, the server 4 can analyze the emotion for each measurement position.

### Modification 1

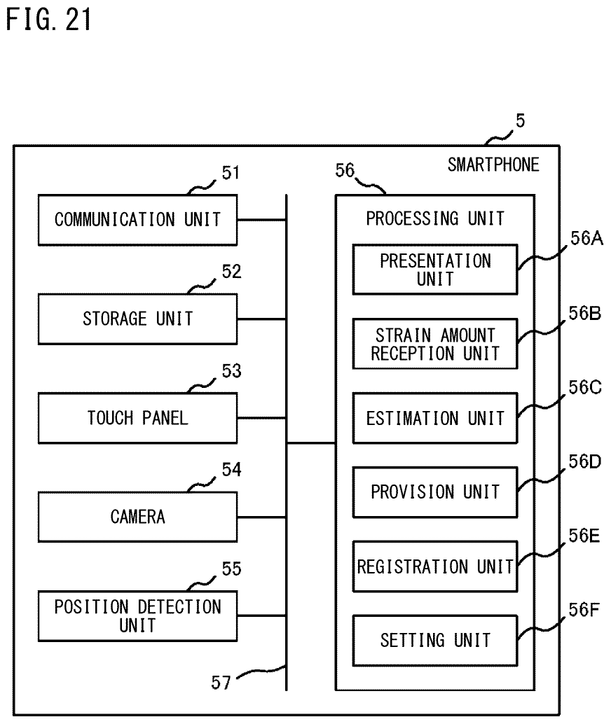

FIG. 21 is a block diagram showing an example of a configuration of the smartphone 5 according to a modification.

The configuration of the smartphone 5 is the same as the configuration of the smartphone 5 according to the embodiment shown in FIG. 6. However, the processing unit 56 further includes a setting unit 56F as a functional processing unit implemented by executing a computer program stored in advance in the storage unit 52.

The setting unit 56F sets, for each sensor ID, whether or not the user 2 permits the provision unit 56D to provide the strain amount to the server 4. For example, in a menu of the emotion analysis application, a button for making a setting for whether or not to permit provision of the strain amount for each sensor ID is provided. By tapping this button, the user 2 makes a setting for whether or not to permit provision of the strain amount, and the setting unit 56F notifies the provision unit 56D of the setting result for whether or not to permit the provision. In a case where the user 2 makes such a setting that does not permit provision of the strain amount, the provision unit 56D stops providing the set data including the strain amount to the server 4 even in a state in which the sensor 3 is in contact with the body surface of the user 2.

On the other hand, in a state in which the sensor 3 is in contact with the body surface of the user 2, if the user 2 changes the setting for provision of the strain amount from a non-permitted state to a permitted state, the provision unit 56D restarts providing the set data including the strain amount to the server 4.

According to the present modification, provision of the strain amount can be stopped by setting, even in a state in which the user 2 has the sensor 3 in contact with the body surface. Thus, it is possible to temporarily interrupt provision of the strain amount by intention of the user 2. In addition, it is also possible to use the sensor 3 as a face sticker without providing the strain amount in the first place.

### Modification 2

In the above embodiment, the strain amount measured by the sensor 3 is transmitted to the server 4 via the smartphone 5. However, the strain amount may be transmitted to the server 4 not via the smartphone 5. For example, an access point is provided for each area at a venue, and the sensor 3 communicates with the access point and transmits the set data of the strain amount and the sensor ID to the access point. The access point transmits the set data received from the sensor 3 to the server 4 via the network 6.

In the server 4, the user information 80 including the sensor IDs and the user IDs is registered. Thus, the server 4 can analyze the emotion of each user on the basis of the set data including the sensor ID registered in the user information 80. On the other hand, in a case where the sensor ID included in the received set data has not been registered in the user information 80, the set data cannot be associated with the user ID. Therefore, the server 4 may discard the set data or may analyze the emotion for each sensor 3 on the basis of the above set data although the user cannot be specified.

In addition, since the server 4 can find the access point through which the set data has passed, the server 4 can analyze the emotions of the users 2 for each access point (i.e., for each area where the access point is placed).

### Additional Note

In the above embodiment, the smartphone 5 does not transmit the time information to the server 4. However, in a case of collectively transmitting the set data received from the sensor 3 to the server 4, the reception times of the set data may be transmitted to the server 4, together with the set data.

A part or the entirety of the components composing the above devices may be formed by a semiconductor device such as one or a plurality of system LSIs. The system LSI is an ultra-multifunctional LSI manufactured by integrating a plurality of components on one chip, and specifically, is a computer system configured to include a microprocessor, a ROM, a RAM, and the like. The RAM stores a computer program. By the microprocessor operating in accordance with the computer program, the system LSI implements its function.

The above computer program may be distributed in a state of being recorded in a computer-readable non-transitory storage medium, e.g., an HDD, a CD-ROM, or a semiconductor memory. The above computer program may be distributed by being transmitted through electric communication lines, wireless/wired communication lines, a network such as the Internet, data broadcasting, or the like.

The server 4 may be formed by a plurality of processors or a plurality of computers.

A part or the entirety of the functions of the server 4 may be provided by cloud computing. That is, a part or the entirety of the functions of the server 4 may be implemented by a cloud server.

The sensor unit 32 may be an electromyogram sensor. The electromyogram sensor is a sensor for measuring slight electric field change (potential difference) occurring in muscle. The electromyogram sensor outputs a current corresponding to the potential difference. The current value outputted from the electromyogram sensor is an example of the physical quantity indicating the degree of change in the shape of the surface (the face surface of the user 2) with which the adhesion surface of the sensor unit 32 contacts.

It should be noted that the embodiment disclosed herein is merely illustrative and not restrictive in all aspects. The scope of the present disclosure is defined by the scope of the claims rather than the above description, and is intended to include meaning equivalent to the scope of the claims and all modifications within the scope.

### REFERENCE SIGNS LIST

1 emotion analysis system (provision system)
2 user
3 sensor
3A adhesion layer
3B circuit layer
3C print layer
3D peel-off sheet
4 server
5 smartphone
6 network
7 wireless base station
31 power supply unit
32 sensor unit
34 wireless communication unit
38 strain gauge
39 storage unit
41 communication unit
42 processing unit
43 storage unit
44 timing unit
51 communication unit
52 storage unit
53 touch panel
54 camera
55 position detection unit
56 processing unit
56A presentation unit
56B strain amount reception unit (physical quantity reception unit)
56C estimation unit
56D provision unit
56E registration unit
56F setting unit
57 internal bus
61 terms of use
62 QR code
72 OK button
73 cancel button
80 user information
90 event information screen
92 OK button
93 cancel button
100 sticker information screen
102A sticker
102B sticker
102C sticker
103A input box for number of sheets
103B input box for number of sheets
103C input box for number of sheets
104 OK button
105 cancel button
110 terms-of-use information screen
111 terms of use
112 OK button
120 QR code reading screen
121 explanatory text
122 icon
130 user ID input screen
131 input box
132 OK button

1. A provision system comprising:
a presentation unit configured to present, to a user, a fact that contacting of a sensor with a body surface of the user serves as a condition for permission to provide a physical quantity to another entity other than the user, the sensor having a contact surface and being configured to measure the physical quantity, the physical quantity indicating a degree of change in a surface with which the contact surface contacts;
an estimation unit configured to estimate a contact state of the sensor with the body surface on the basis of the physical quantity; and
a provision unit configured to start providing the physical quantity to the other entity, on the basis of an estimation result for the contact state by the estimation unit.

2. The provision system according to claim 1, wherein
the estimation unit estimates that the sensor is in contact with the body surface, on the basis of a comparison result between the physical quantity and a first threshold, and
when the estimation unit has estimated that the sensor is in contact with the body surface, the provision unit starts providing the physical quantity to the other entity.

3. The provision system according to claim 2, wherein
the estimation unit estimates that the sensor is in contact with the body surface, on the basis of a period in which the comparison result between the physical quantity and the first threshold continues.

4. The provision system according to claim 1, wherein
the estimation unit estimates that the sensor is not in contact with the body surface, on the basis of a comparison result between the physical quantity and a second threshold, and
when the estimation unit has estimated that the sensor is not in contact with the body surface, the provision unit finishes providing the physical quantity to the other entity.

5. The provision system according to claim 4, wherein
the estimation unit estimates that the sensor is not in contact with the body surface, on the basis of a period in which the comparison result between the physical quantity and the second threshold continues.

6. The provision system according to claim 1, further comprising:
a registration unit configured to register a sensor identifier for identifying the sensor; and
a physical quantity reception unit configured to receive, from the sensor, a set of the physical quantity and the sensor identifier of the sensor, wherein
the provision unit provides, to the other entity, the set including the sensor identifier registered by the registration unit among the sets received by the physical quantity reception unit.

7. The provision system according to claim 6, wherein
the provision unit, further, does not transmit, to a device used by the other entity, the set not including the sensor identifier registered by the registration unit among the sets received by the physical quantity reception unit.

8. The provision system according to claim 6, wherein
the registration unit, further, provides a user identifier for identifying the user and the sensor identifier, to a device used by the other entity.

9. The provision system according to claim 1, wherein
the provision unit, further, provides measurement position information for the physical quantity, to a device used by the other entity.

10. The provision system according to claim 1, further comprising a setting unit configured to make a setting for whether or not the user permits providing the physical quantity, wherein
when the user does not permit providing the physical quantity, the provision unit stops providing the physical quantity to the other entity.

11. A provision method comprising:
a presentation step of presenting, to a user, a fact that contacting of a sensor with a body surface of the user serves as a condition for permission to provide a physical quantity to another entity other than the user, the sensor having a contact surface and being configured to measure the physical quantity, the physical quantity indicating a degree of change in a surface with which the contact surface contacts;
an estimation step of estimating a contact state of the sensor with the body surface on the basis of the physical quantity; and
a provision step of starting providing the physical quantity to the other entity, on the basis of an estimation result for the contact state.

12. A non-transitory computer readable storage medium storing a computer program for causing a computer to function as:
a presentation unit configured to present, to a user, a fact that contacting of a sensor with a body surface of the user serves as a condition for permission to provide a physical quantity to another entity other than the user, the sensor having a contact surface and being configured to measure the physical quantity, the physical quantity indicating a degree of change in a surface with which the contact surface contacts;
an estimation unit configured to estimate a contact state of the sensor with the body surface on the basis of the physical quantity; and
a provision unit configured to start providing the physical quantity to the other entity, on the basis of an estimation result for the contact state by the estimation unit.
